(19) United States
(12) Patent Application Publication
(10) Pub. No.: US 2013/0150670 A1
(43) Pub. Date: Jun. 13, 2013

(54) THORACIC SCOPE PORT CLEANER

(71) Applicant: Covidien LP, Mansfield, MA (US)

(72) Inventors: Cormac O'Prey, Hertfordshire (GB); Valerie Anne Scott, Cambridge (GB); Alistair Ian Fleming, Cambridge (GB)

(73) Assignee: Covidien LP, Mansfield, MA (US)

(21) Appl. No.: 13/707,173

(22) Filed: Dec. 6, 2012

Related U.S. Application Data
(60) Provisional application No. 61/567,896, filed on Dec. 7, 2011.

(51) Int. Cl. A61B 1/00

(52) U.S. Cl. CPC A61B 1/00137
USPC 600/127

(57) ABSTRACT

An instrument for cleaning a lens of a scope includes an elongated sheath and a cleaning portion. The elongated sheath defines a lumen dimensioned and configured to slidingly receive the scope therein. The cleaning portion is positioned in a distal portion of the lumen. The cleaning portion includes a membrane formed from an elastic material. The membrane includes one or more openings to facilitate translation of the scope through the membrane.

### CROSS-REFERENCE TO RELATED APPLICATIONS

This application claims priority to, and the benefit of, U.S. Provisional Patent Application Ser. No. 61/567,896, filed on Dec. 7, 2011, the entire contents of which are incorporated by reference herein.

### BACKGROUND

1. Technical Field

The present disclosure relates to a cleaning apparatus configured to remove contaminants, e.g., debris and/or moisture, from the lens of a minimally invasive thoracic viewing instrument.

2. Background of Related Art

Minimally invasive surgery has become increasingly popular in recent years. Minimally invasive surgery eliminates the need to cut a large incision in a patient, thereby reducing discomfort, recovery time, and many of the deleterious side effects associated with traditional open surgery. Minimally invasive viewing instruments, e.g., laparoscopes and endoscopes, are optic instruments to facilitate the viewing of internal tissues and/or organs.

Laparoscopic surgery involves the placement of a laparoscope in a small incision in the abdominal wall of a patient to view the surgical site. Endoscopic surgery involves the placement of an endoscope in a naturally occurring orifice, e.g., mouth, nose, anus, urethra, and vagina to view the surgical site. Other minimally invasive surgical procedures include video assisted thoracic surgery and cardiovascular surgery conducted through small incisions between the ribs. These procedures also utilize scopes to view the surgical site.

A typical minimally invasive viewing instrument, e.g., a laparoscope or an endoscope, includes a housing, an elongated lens shaft extending from one end of the housing, and a lens that is provided in the distal end of the lens shaft. A camera viewfinder extends from the other end of the housing. A camera is connected to the housing and transmits images of the surgical field viewed through the lens to a monitor on which the images are displayed. During a surgical procedure, the distal end portion of the lens shaft is extended into the patient, while the proximal end portion of the lens shaft, the housing and the camera viewfinder remain outside the patient. In this manner, the laparoscope/endoscope is positioned and adjusted to view particular anatomical structures in the surgical field on the monitor.

During insertion of an endoscope or a laparoscope into the body and during the surgical procedure, debris, e.g., organic matter and moisture, may be deposited on the lens of the endoscope. The buildup of debris and condensation on the lens impairs visualization of the surgical site, and often necessitates cleaning of the lens.

### SUMMARY

The present disclosure is generally related to an instrument for cleaning the lens of a medical viewing instrument, such as an endoscope, during a minimally invasive surgical procedure.

In one aspect, an instrument for cleaning the lens of a scope includes an elongated sheath having a lumen dimensioned and configured to slidingly receive a scope therein. A cleaning portion is positioned in the lumen of the sheath at the distal portion. The cleaning portion includes a membrane formed from an elastic material. One or more openings in the membrane facilitate distal translation of the scope through the sheath.

As the scope is distally translated, the lens passes by and slidably engages the cleaning portion to facilitate removal of debris and/or moisture.

In one embodiment, the cleaning portion includes a cleaning member integrally formed with the sheath as a unitary structure. Integrally forming the cleaning member as a unitary structure may reduce manufacturing costs, enhance its durability, and inhibit leakage of fluids and/or gases through the tube of the surgical instrument.

The membrane may include a plurality of flaps. In some embodiments, the flaps substantially obstruct the lumen at the distal portion of the sheath in a first position, and deflect outwardly to a second position in response to axial distal translation of the scope through the lumen. In some embodiments, the flaps may be biased toward a closed position. The cleaning member may include one or more slits formed therein that define the one or more flaps. The flaps can translate across and away from the lens of the scope in response to the distal axial translation of the scope through the lumen of the sheath. The flaps may approximate a surface of the lens of the scope, thereby facilitating cleaning of the lens as the scope is translated in an axial distal direction through the lumen of the tube.

In some embodiments, the cleaning member may be formed from an elastic or flexible material that includes a small centrally disposed opening adapted for the reception of the lens therethrough. In a first position, the opening can be substantially closed. In such embodiments, as the scope distally translates through the tube, the scope can press against and expand the opening, and as the opening expands, the lens of the scope is pressed against the surface of the cleaning member facilitating the removal of debris and/or moisture from the lens of the scope.

In another embodiment, the sheath may include one or more internal threads at the distal portion that matingly engage one or more external threads at a proximal end of the connecting portion of the cleaning portion. Alternatively, the sheath may include one or more external threads at the distal portion that matingly engage one or more internal threads at a proximal end of the connecting portion of the cleaning portion. The threads can facilitate releasable securement of the cleaning portion to the sheath.

It is contemplated that in each of the presently disclosed embodiments, the cleaning member may be impregnated with a cleaning material.

In some embodiments, the membrane is composed of a plurality of materials having different properties. In some embodiments, an inner layer is softer than an outer layer.

These and other features of the present disclosure will be more fully described with reference to the appended figures.

### BRIEF DESCRIPTION OF THE DRAWINGS

By way of description only, embodiments of the present disclosure will be described herein with reference to the accompanying drawings, in which:

FIG. 1 is a perspective view of an instrument in accordance with an embodiment of the present disclosure with the scope in a retracted position;

FIG. 2A is a perspective view of a distal portion of the instrument of FIG. 1 shown in a first condition corresponding to the position of FIG. 1;

FIG. 2B is a perspective view of the distal portion of the instrument of Fig. I shown in a second condition with the scope lens advanced through the cleaner;

FIG. 3A is a perspective view of a distal portion of an instrument in accordance with another embodiment of the present disclosure shown in a first condition;

FIG. 3B is a perspective of the distal portion of the instrument of FIG. 3A shown in a second condition with the scope lens advanced through the cleaner;

FIG. 4A is a perspective view of a distal portion of an instrument in accordance with another embodiment of the present disclosure shown in a first condition;

FIG. 4B is a side view in partial cross-section of the distal portion of the instrument of FIG. 4A;

FIG. 4C is an exploded perspective view of the distal portion of the instrument of FIG. 4A; FIG. 5A is a perspective view of a distal portion of an instrument in accordance with yet another embodiment of the present disclosure shown in a first condition; and

FIG. 5B is an exploded perspective view of the distal portion of the instrument of FIG. 5A.

### DETAILED DESCRIPTION

Particular embodiments of the present disclosure will be described with reference to the accompanying drawings. In the figures and in the description that follow, in which like reference numerals identify similar or identical elements, the term “proximal” will refer to the end of the instrument that is closer to the operator during use, while the term “distal” will refer to the end that is farther from the operator during use.

An endoscope typically includes an endoscope housing or body which can be rigid or flexible, depending on its surgical application. A camera viewfinder, e.g. an eyepiece, is located at a proximal (imaging) end of the scope housing. A lens is provided at the distal end of the scope body.

In typical use of the endoscope, the viewfinder is adapted to sight images of a surgical field in the patient, e.g. an abdominal cavity, thoracic cavity, etc., as the position of the scope is adjusted to view a particular anatomical structure or structures in the surgical field. The camera is adapted to receive images of the surgical field sighted through the lens and transmit the images to an external monitor that is connected to the camera and on which the images of the surgical field are displayed. That is, a visual display device is operatively connected to the eyepiece to convert the optical signal into a video signal to produce a video image on the monitor (or for storage on select media). Accordingly, the monitor enables a surgical team to view the anatomical structure or structures in the surgical field inside the patient as the surgical procedure is carried out using minimally invasive or endoscopic surgical instruments. Throughout the surgical procedure, biological tissue or matter has a tendency to contact and build up on the lens of the scope. This tends to obscure the images of the surgical field as they are displayed on the monitor.

The present disclosure includes wiping flaps to clean the lens of the scope during the surgical procedure to maintain a clear image without having to remove the scope from the patient's body.

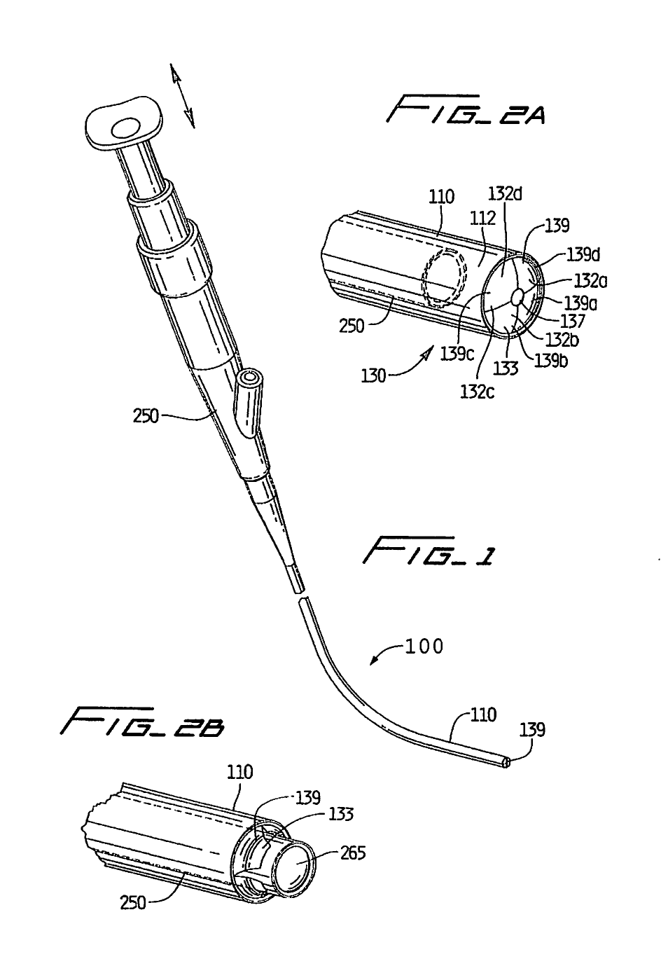

A first embodiment of a minimally invasive surgical instrument 100 that is configured and adapted to clean, i.e., remove debris and/or moisture, from a viewing portion 265, e.g., a lens, of a viewing instrument 250 will now be described with reference to FIGS. 1-2B. The surgical instrument 100 includes a tube or sheath 110 having a lumen 112 that is configured and adapted to receive a scope 250 therethrough. In particular, the scope 250 is axially translatable through a distal portion 130 of the sheath 110. A cleaning portion 139 is positioned in the distal portion 130 of the sheath 110. Translation of the scope 250 through the distal portion 130 of the sheath 110 results in interaction between the viewing portion 265 of the scope 250 and the cleaning portion 139. This interaction facilitates removal of debris and/or moisture, or other substances that may obstruct the viewing portion 265. The operation of the cleaning portion 139 may be performed with the surgical instrument 100 in situ, i.e., within a patient's body cavity, during a surgical procedure so that the scope 250 does not need to be removed from the patient, thereby resulting in cleaning of the viewing portion 265 with minimal disruption to the surgical procedure.

The scope can be inserted into an already placed sheath or alternatively positioned within the sheath and together inserted into the body. The sheath can accommodate various types of scopes, including but not limited to laparoscopes, thoracic scopes, etc. For example, during video assisted thoracoscopic surgery, a thoracic port is inserted through the ribs to provide access to the thoracic cavity for access to the lung or other tissue. A separate access is provided through the ribs to insert a scope to visualize the thoracic cavity during the surgical procedure. The sheath of the present disclosure can be utilized with the thoracic scope to maintain a clean lens to provide consistent visibility and imaging during the surgical procedure. The sheath can also be utilized with a flexible scope if composed of a sufficiently flexible material.

As shown in FIGS. 2A and 2B, the cleaning portion 139 includes a cleaning member 133. The cleaning member 133 may be a membrane, formed from a thin, pliable material that obstructs passage of an object therethrough in a first position, and facilitates passage of an object therethrough in a second position. To facilitate passage of an object through the cleaning member 133, the cleaning member 133 includes one or more slits 132a-d that define one or more flaps 139a-d. As shown in FIG. 2A, with the scope 250 retracted within sheath 110, the flaps 139a-d substantially obstruct the path of the scope 250 through the distal portion 130 of the sheath 110. The cleaning member 133 may also include a small opening 137 centrally disposed with the cleaning member 133 and between the one or more flaps 139a-d. The opening 137 provides a stress concentration and facilitates axial translation of the scope 250 between the one or more flaps 139a-d of the cleaning member 133. The flaps are biased toward an approximated position.

The cleaning portion 139 may be formed from a flexible or compliant material that is configured and adapted to approximate the contour of the viewing portion 265 of the scope 250. The cleaning portion 139 includes a cleaning member 133 that is biased toward the first condition shown in FIG. 2A. As described above, the cleaning member 133 may be formed from an elastic membrane, i.e., a thin layer of material, that substantially obstructs the path of the scope 250 distally through the sheath 110 in a first condition and parts, in a second condition, to accommodate continued distal translation of the lens 250.

In the first condition, flaps 139a-d are positioned against one another and substantially obstruct the path of the scope 250 through the distal portion 130 of the sheath 110. The cleaning portion 139 substantially approximates the contours of the viewing portion 265 as the scope 250 is distally translated through the cleaning portion 139.

The cleaning portion 139 may be frictionally fit within the sheath 110. In particular, the cleaning portion 139 may have a diameter larger than that defined by sheath 110 such that placement of the cleaning portion 139 within the sheath 110 frictionally secures the cleaning portion 139 within the sheath 110 due to the frictional engagement of the sheath inner wall.

As the scope 250 is distally and axially translated through the tube 110, the viewing portion 265 and the flaps 139a-d interact to remove debris from the surface of the viewing portion 265 as the viewing portion 265 is passed through and engaged by the flaps 139a-139d. The viewing instrument 250 is distally translated through the sheath 110 until the flaps 139a-d are parted and do not obstruct the viewing portion 265. With the scope 250 fully extended through the distal portion 130 of the sheath 110, the inward bias of the flaps 139a-d against the exterior of the scope 250 (FIG. 2B) may facilitate stabilization of the scope 250. Moreover, the cleaning member 133 may include a cleaning solution and/or material. For example, the cleaning solution and/or material may be impregnated into the cleaning member 133.

In one embodiment, the cleaning member 133 may include multiple layers of material. Each layer of material may have different properties. For example, the outer layer of material may be stiffer than the inner layer of material. By having a softer inner layer of material that contacts the viewing portion 265 of the scope 250, damage, e.g., scratching, of the viewing portion 265 may be inhibited. In addition, a stiffer outer layer of material may facilitate protection of the viewing portion 265 when scope 250 is retracted within the sheath 110. The stiffer material can also add some rigidity to the flaps to bias them to a closed position.

The use and operation of the surgical instrument 100 will now be described with reference to FIGS. 2A and 2B. During use of the surgical instrument 100, the viewing portion 265 of the scope 250 extending beyond the distal end of sheath 112, may become obstructed by debris and/or moisture. The viewing portion 265 may be cleaned in situ, i.e., during the surgical procedure, without removing the surgical instrument 100 from the surgical site. In particular, the scope 250 is retracted to be positioned proximal to the cleaning member 133 to a position shown in FIG. 2A. Then, the scope 250 is distally translated through the cleaning member 133. The distal translation of the scope 250 through the cleaning member 133 forces the viewing portion 265 against the flaps 139a-d, thereby resulting in cleaning of the viewing portion 265 and parting of the flaps 139a-d. In this second condition, as shown in FIG. 2B, the flaps 139a-d are parted and do not obstruct the viewing portion 265 of the scope 250. During the surgical procedure, these steps may be repeated as needed to clean the viewing portion 265.

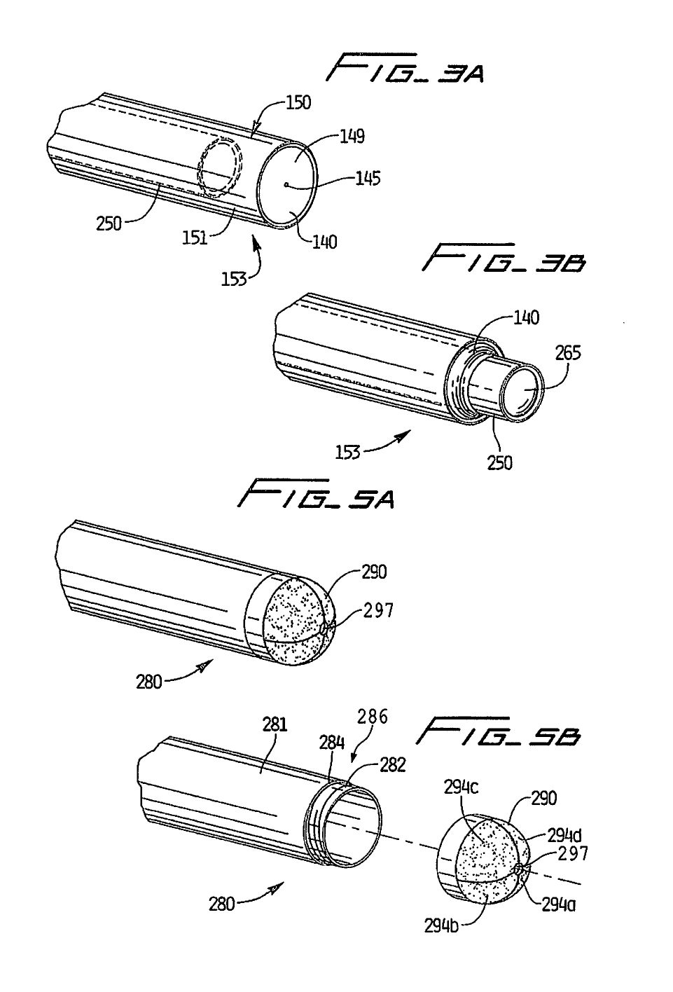

In another embodiment shown in FIGS. 3A and 3B, a surgical instrument includes a sheath 151 with a cleaning portion 149. The surgical instrument 150 is substantially similar to the surgical instrument 100 except in the following respects, as will now be described hereinbelow. In particular, the surgical instrument 150 includes a sheath 151 and a cleaner portion that includes a cleaner member in the form of an elastic membrane 140. The elastic membrane 140 includes a centrally disposed opening 145. The elastic membrane 140 can be frictionally retained within distal portion 153 of sheath 151. In the first condition (FIG. 3A), the opening 145 is substantially closed. As the scope 250 is distally translated through the lumen of sheath 151, the viewing portion 265 of the scope 250 is pressed against the elastic membrane 140, thereby cleaning the viewing portion 265. Continued distal translation of the scope 250 within sheath 151 expands the dimension of the opening 145 until the scope 250 is passed through the opening 145 and the membrane 140 is positioned around the scope 250 (FIG. 3B). Translation of the scope 250 through the sheath 151 and the transitioning of the surgical instrument between the first and second conditions (FIGS. 3A-B), effects the cleaning of the viewing portion 265 of scope 250. Similar to the cleaning member 133, the membrane 140 may also include a cleaning solution and/or material, e.g., the membrane 140 may be impregnated with a cleaning solution and/or material.

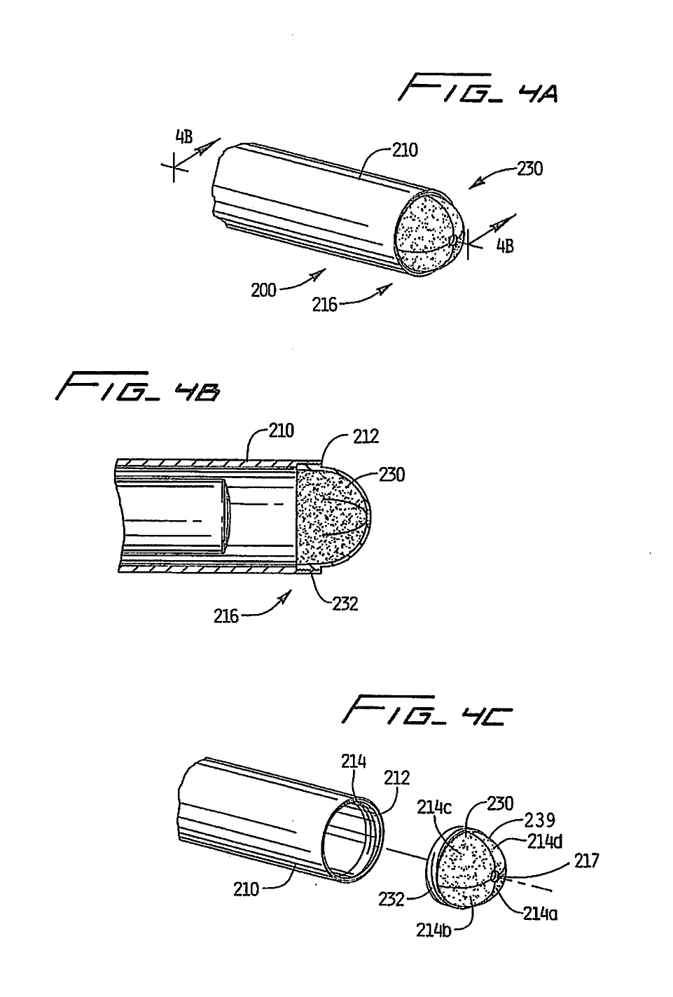

In the foregoing embodiments, the cleaning member can be frictionally retained within the sheath. Alternatively, the sheath and the cleaning member may be matingly engaged to one another. In particular, a surgical instrument 200 that is substantially similar to surgical instrument 100, except in the following respects, will now be described with reference to FIGS. 4A-4C. The surgical instrument 200 includes a sheath 210 having a threading 212 along an interior surface 214 at the distal portion 216 of the sheath 210. A cleaning portion 230 having a cleaning member 239 is provided that is substantially similar to the cleaning portion 139 of FIG. 2A. The cleaning portion 230 includes a complementary exterior threading 232 at a proximal portion thereof. The threading 232 and the threading 212 are configured and adapted to matingly engage one another in a secure manner that may be releasable. In this manner, the cleaning portion 230 can be attached to the sheath 210. The cleaning member 239 includes flaps 214a-214d which define opening 217. Flaps 214a-214d and opening 217 are substantially identical to the flaps 132a-132d and opening 137 of the embodiment shown in FIGS. 2A and 2B, respectively.

In an alternative embodiment, shown in FIGS. 5A and 5B, a surgical instrument 280 includes threading 282 along an exterior 284 of the sheath 281 at a distal portion 286 thereof. A cleaning portion 290 having a cleaning member is substantially similar to the cleaning portion 139 of FIG. 2A except in the following respect. The cleaning portion 290 includes a threading (not shown) along an interior proximal surface. The threading is configured and adapted to matingly engage threading 282, and can be releasable. In this manner, the cleaner portion 290 can be attached to sheath 281. Cleaning portion 290 has an opening 297 and flaps 294a, 294b, 294c and 294d substantially identical to opening 137 and flaps 132a-132d of FIGS. 2A, 2B, respectively, to clean the scope 250.

Fluid conduit(s) can be provided in the foregoing sheaths to apply a cleaning fluid.

The flaps can be symmetrically or asymmetrically arranged. Additionally, an integral hinge, spring or other mechanism can be attached to the flaps to hold the flaps against the lens during re-insertion of the scope.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An instrument for cleaning a lens of a scope, comprising:
an elongated sheath having a proximal portion and a distal portion, the sheath defining a lumen extending longitudinally therethrough, the lumen dimensioned and configured to slidingly receive the scope therein; and
a cleaning portion positioned in a distal portion of the lumen, the cleaning portion including a membrane formed from an elastic material, the membrane including one or more openings to facilitate translation of the scope through the membrane.

2. The instrument of claim 1, wherein the membrane includes a plurality of flaps.

3. The instrument of claim 2, wherein the plurality of flaps substantially obstruct the lumen at the distal portion of the sheath in a first position.

4. The instrument of claim 2, wherein the plurality of flaps deflect outwardly to a second position in response to axial distal translation of the scope through the lumen.

5. The instrument of claim 2, wherein the one or more openings is a single substantially centrally disposed opening.

6. The instrument of claim 5, wherein the single centrally disposed opening is substantially closed in a first position and is enlarged in response to distal translation of the scope through the opening.

7. The instrument of claim 1, wherein the cleaning portion includes a connecting portion connectable to the sheath.

8. The instrument of claim 7, wherein the connecting portion includes one or more threads and the distal portion of the sheath includes one or more threads, the one or more threads of the connecting portion matingly engageable with the one or more threads of the sheath.

9. The instrument of claim 2, wherein the plurality of flaps approximate a surface of the lens of the scope.

10. The instrument of claim 1, wherein the cleaning portion frictionally engages an internal wall of the sheath.

11. The instrument of claim 4, wherein the plurality of flaps are biased toward the first position.

12. The instrument of claim 1, wherein the cleaning portion includes a plurality of slits defining a plurality of flaps.

13. The instrument of claim 1, wherein the cleaning portion includes a cleaning material.

14. The instrument of claim 1, wherein the membrane is composed of a plurality of materials having different properties.

15. The instrument of claim 14, wherein an inner layer of the membrane is softer than an outer layer of the membrane.

16. The instrument of claim 1, wherein the cleaning portion is a separate unit removably attached to the sheath.
